(19) United States
(12) Patent Application Publication
(10) Pub. No.: US 2013/0158290 A1
(43) Pub. Date: Jun. 20, 2013

(54) PRODUCTION OF OXYGENATED COMPOUNDS IN THE PRESENCE OF A CATALYST SUSPENDED IN AN INERT LIQUID

(71) Applicants: Stephane MARIE-ROSE, Sherbrooke (CA); Esteban CHORNET, Sherbrooke (CA)

(72) Inventors: Stephane MARIE-ROSE, Sherbrooke (CA); Esteban CHORNET, Sherbrooke (CA)

(21) Appl. No.: 13/675,094

(22) Filed: Nov. 13, 2012

Related U.S. Application Data
(60) Provisional application No. 61/562,828, filed on Nov. 22, 2011.

(51) Int. Cl. B01J 29/04; C07C 41/01; C07C 45/61; C07C 67/36

(52) U.S. Cl. CPC B01J 29/04; C07C 67/36; C07C 41/01; C07C 45/61
USPC 560/265; 568/671; 568/484

(57) ABSTRACT

A method of producing at least one oxygenated compound such as methyl acetate, dimethyl ether, and formaldehyde, by reacting dimethyl carbonate with carbon monoxide in the presence of at least one solid catalyst, such as a zeolite catalyst, suspended in an inert liquid, such as an inert oil.

This application claims priority based on provisional Application Ser. No. 61/562,828, filed Nov. 22, 2011, the contents of which are incorporated by reference in their entirety.

This invention relates to the production of oxygenated compounds, such as methyl acetate, dimethyl ether, and formaldehyde, from dimethyl carbonate and carbon monoxide. More particularly, this invention relates to the production of the above-mentioned oxygenated compounds by reacting dimethyl carbonate and carbon monoxide in the presence of at least one solid catalyst suspended in an inert liquid.

In general, methyl acetate may be produced by reacting methanol with carbon monoxide in the presence of a catalyst and a co-catalyst, which may be methyl iodide or other halogenated compounds. An example of a catalyst and co-catalyst is a rhodium catalyst with a halide promoter, such as those described is published U.S. Patent Application No. US2009/0326080.

Alternatively, methyl acetate can be produced by reacting dimethyl carbonate with carbon monoxide in the presence of a mordenite zeolite catalyst. Examples of mordenite zeolite catalysts which may be used to convert dimethyl carbonate to methyl acetate are disclosed in PCT Application No. WO2008/132442, and published U.S. Application Nos. US2010/0267985. US2010/0274045, US2010/0311567, US2010/0317888.

In accordance with an aspect of the present invention, there is provided a method of producing at least one oxygenated compound selected from the group consisting of methyl acetate, dimethyl ether, and formaldehyde, and mixtures thereof. The method comprises reacting dimethyl carbonate with carbon monoxide to convert at least a portion of the dimethyl carbonate and the carbon monoxide to the at least one oxygenated compound or mixtures thereof. The dimethyl carbonate and the carbon monoxide are reacted in the presence of at least one solid catalyst suspended in an inert liquid.

In a non-limiting embodiment, the at least one solid catalyst is at least one zeolite catalyst.

Zeolite catalysts which may be employed in accordance with the present invention include, but are not limited to, faujasite zeolites, zeolite Beta, Linde Type L (LTL) zeolite, MCM-41, mordenite zeolites, zeolite X, zeolite Y, and ZSM-5 or mixtures thereof.

In a non-limiting embodiment, the at least one zeolite is a faujasite zeolite. In another non-limiting embodiment, the zeolite is zeolite Beta.

In yet another non-limiting embodiment, the zeolite is LTL zeolite. In another non-limiting embodiment, the zeolite is MCM-41 zeolite.

In a non-limiting embodiment, the above-mentioned zeolites are subjected to cation exchange with at least one cation. In a non-limiting embodiment, the zeolite is subjected to cation exchange with at least one cation selected from the group consisting of sodium, lithium, cesium, or mixtures thereof.

In another non-limiting embodiment, the above-mentioned zeolites may be mixed with alumina.

In a non-limiting embodiment, the at least one zeolite catalyst may be used as a support for at least one catalytic metal. Such catalytic metals include metals that contribute to the non-dissociative insertion of carbon monoxide. In a non-limiting embodiment, the catalytic materials include, but are not limited to, transition metals of Group VII and Group VIII of the periodic table.

As mentioned hereinabove, the at least one solid catalyst is suspended in an inert liquid. In a non-limiting embodiment, the inert liquid is an inert oil. In a non-limiting embodiment, the insert oil is as a white mineral oil, such as, for example, Witco-70 or Drakeol.

In a non-limiting embodiment, the at least one oxygenated compound is methyl acetate. In another non-limiting embodiment, the at least one oxygenated compound is dimethyl ether. In yet another non-limiting embodiment, the at least one oxygenated compound is formaldehyde.

In a further non-limiting embodiment, the at least one oxygenated compound is at least two of methyl acetate, dimethyl ether, and formaldehyde. In still another non-limiting embodiment, each of methyl acetate, dimethyl ether, and formaldehyde is produced by reacting dimethyl carbonate with carbon monoxide.

In another non-limiting embodiment, the carbon monoxide is obtained from synthesis gas. Thus, in a non-limiting embodiment, the dimethyl carbonate is contacted with synthesis gas, whereby the dimethyl carbonate is reacted with the carbon monoxide in the synthesis gas to produce at least one of methyl acetate, dimethyl ether, formaldehyde, or mixtures thereof.

In a non-limiting embodiment, the dimethyl carbonate is in the form of a gas when it is reacted with carbon monoxide gas to produce at least one methyl acetate, dimethyl ether, and formaldehyde.

In a non-limiting embodiment, the dimethyl carbonate is reacted with the carbon monoxide at a temperature of from about 100° C. to about 600° C. In another non-limiting embodiment, the dimethyl carbonate is reacted with the carbon monoxide at a temperature of from about 100° C. to about 400° C.

In another non-limiting embodiment, the dimethyl carbonate and the carbon monoxide are reacted at a pressure of from about 14 psi to about 900 psi.

In a non-limiting embodiment, the dimethyl carbonate is reacted with the carbon monoxide at a molar ratio of dimethyl carbonate to carbon monoxide of from about 0:25:1 to about 10:1. In another non-limiting embodiment, the dimethyl carbonate is reacted with the carbon monoxide at a molar ratio of dimethyl carbonate to carbon monoxide of from about 0:25:1 to about 2:1. In another non-limiting embodiment. the dimethyl carbonate is reacted with the carbon monoxide at a molar ratio of dimethyl carbonate to carbon monoxide of from about 0.5:1 to about 1:1.

In a non-limiting embodiment, the dimethyl carbonate is reacted with the carbon monoxide at a gas hourly space velocity (GHSV), based on the amount of carbon monoxide present, of from about 1000 h⁻¹ to about 60,000 h⁻¹.

In a non-limiting embodiment, the dimethyl carbonate is reacted with carbon monoxide gas in a suitable reactor containing the solid catalyst suspended in an inert liquid, such as an inert oil, for example. In such a non-limiting embodiment, the reactor generally is referred to as a “three-phase reactor”.

In a non-limiting embodiment, the catalyst s in the form of a powder which is suspended in an inert high boiling oil, such as a white mineral oil such as Witco-70 or Drakeol. In a non-limiting embodiment, the dimethyl carbonate is vaporized, and then mixed with carbon monoxide gas. A combined stream of vaporized dimethyl carbonate and carbon monoxide gas is dissolved in the oil, and the dissolved molecular species are reacted on the catalytic surfaces of the slurried catalyst. The reactor may be operated under the temperature and pressure conditions hereinabove described.

Although the scope of the present invention is not to be limited to any theoretical reasoning, Applicants have discovered that the reaction of dimethyl carbonate with carbon monoxide gas in the presence of a solid catalyst suspended in an inert liquid in a “three-phase” reactor provides for better control of the reaction temperature, improved heat exchange when compared with a fixed bed reactor, and the ability to remove the catalyst during the reaction without shutdown of the reactor. Also, the “three-phase” reactor provides for a reduction in mass transfer limitations associated with diffusion between reactants and the catalyst.

Although the scope of the present invention is not intended to be limited to any theoretical reasoning, the “three-phase” reactor is used to adsorb heat released during the reaction and thus prevent potential “hot spots” due to overheating, and consequently, catalyst deactivation.

In general, the catalyst has a particle size that is small enough for external mass transfer and internal diffusion resistance. In a non-limiting embodiment, the catalyst has a particle size of from about 20 microns to about 120 microns. In anther non-limiting embodiment, the catalyst has a particle size of from about 20 microns to about 80 microns.

The invention now be described with respect to the drawings, wherein:

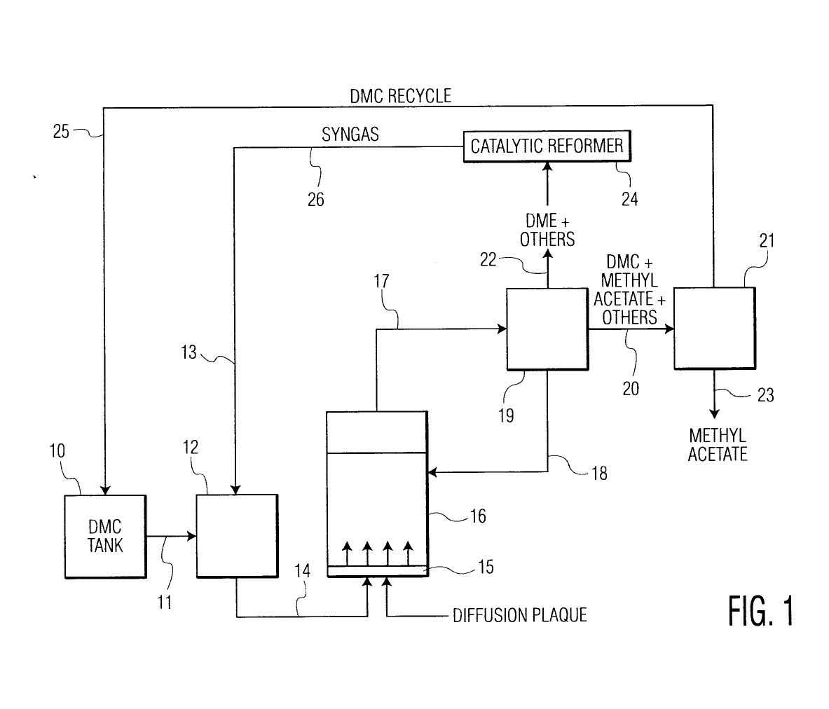

FIG. 1 is a schematic of a non-limiting embodiment of the process of the present invention; and

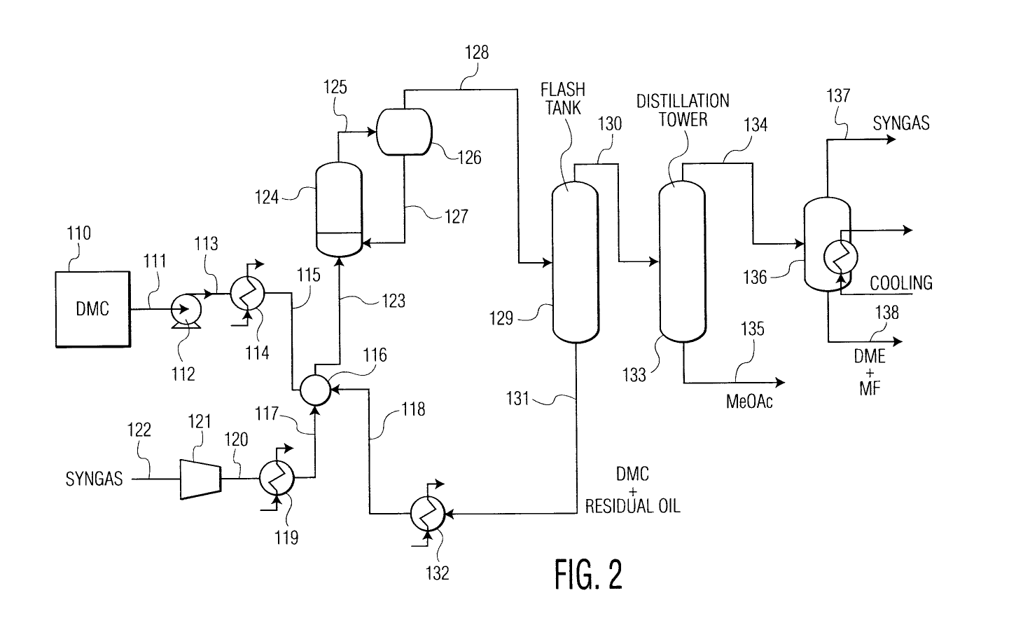

FIG. 2 is a schematic of another non-limiting embodiment of the process of the present invention.

Referring now to the drawing, as shown in FIG. 1, a liquid feed of dimethyl carbonate (DMC) in tank 10 is passed from line 11 to evaporator 12, wherein the liquid dimethyl carbonate is vaporized into gaseous dimethyl carbonate. In general, the evaporator is operated at a temperature of from about 90° C. to about 200° C. As the dimethyl carbonate is evaporated in evaporator 12, synthesis gas is passed to evaporator 12 from line 13, and the vaporized dimethyl carbonate and the synthesis gas are mixed in evaporator 12. A combined stream of dimethyl carbonate and synthesis gas then is withdrawn from evaporator 12 through line 14, and then is passed through diffusion plaque 15, which is contained within “three phase” reactor 16. Reactor 16 contains a solid catalyst, such as a zeolite catalyst, which is suspended in an inert liquid, such as an inert oil.

In reactor 16, the vaporized dimethyl carbonate and the synthesis gas are reacted under conditions such as those hereinabove described such that the carbon monoxide in the synthesis gas is reacted with the dimethyl carbonate to form oxygenated compounds such as methyl acetate, dimethyl ether (DME), and formaldehyde. A product containing methyl acetate, dimethyl ether, formaldehyde, unreacted dimethyl carbonate, carbon dioxide, unreacted synthesis gas and a portion of the suspension of catalyst in oil, is withdrawn from reactor 16 through line 17, and is passed to flash tower 19. In general, the flash tower is operated at a temperature of from about 5° C. to about 20° C., and at a pressure of from about 14 psi to about 900 psi. Flash tower 19 is operated under conditions such that the methyl acetate and unreacted dimethyl carbonate are separated from the dimethyl ether, formaldehyde, carbon dioxide, and unreacted synthesis gas.

Methyl acetate and unreacted dimethyl carbonate are withdrawn from flash tower 19 through line 20 and passed to distillation column 21. The suspension of catalyst in oil is withdrawn from line 18 and recycled to reactor 16. In distillation column 21, a methyl acetate product is separated from the dimethyl carbonate and recovered through line 23. The dimethyl carbonate is withdrawn from distillation column 21 through line 25 as a liquid. The dimethyl carbonate in line 25 then is recycled to tank 10, wherein the recycled dimethyl carbonate is mixed with fresh dimethyl carbonate and is withdrawn from tank 10 through line 11 and passed to evaporator 12.

Dimethyl ether, formaldehyde, carbon dioxide, and unreacted synthesis gas are withdrawn from flash tower 19 through line 22, and passed to catalytic reformer 24. In catalytic reformer 24, the dimethyl ether, formaldehyde, and carbon dioxide which are passed to the catalytic reformer, are subjected to catalytic reforming conditions in order to produce additional synthesis gas. The reaction of dimethyl ether, formaldehyde, and carbon dioxide in catalytic reformer 24 is effected in the presence of an appropriate reforming catalyst. Such catalysts include, but are not limited to, nickel-based catalysts and noble metal based catalysts. In general, the catalytic reformer is operated at a temperature of from about 300° C. to about 900° C., and at a pressure around atmospheric pressure. The synthesis gas then is withdrawn from catalytic reformer 24 through line 26. The synthesis gas in line 26 then is passed to line 13, and is recycled to evaporator 12.

In another non-limiting embodiment, as shown in FIG. 2, a liquid feed of dimethyl carbonate (DMC) in tank 110 is passed from line 111, through pump 112, and line 113 to evaporator 114, wherein the liquid dimethyl carbonate is vaporized. In general, evaporator 114 is operated at a temperature of from about 90° C. to about 200° C. The gaseous dimethyl carbonate is withdrawn from evaporator 114 through line 115, and is passed to mixing chamber 116. As the dimethyl carbonate is evaporated in evaporator 114, synthesis gas from line 122 is compressed in compressor 121. The compressed synthesis gas is withdrawn from compressor 121 through line 120, and is passed to preheater 119. The heated synthesis gas is withdrawn from preheater 119 through line 117 and is passed to mixing chamber 116. The vaporized dimethyl carbonate and synthesis gas are mixed in mixing chamber 116, and then withdrawn from mixing chamber 116 through line 123, and passed to “three phase” reactor 124. Reactor 124 contains a solid catalyst, such as a zeolite catalyst, which is suspended in an inert liquid, such as an inert oil.

In reactor 124, the vaporized dimethyl carbonate and the synthesis gas are reacted under conditions such as those hereinabove described such that the carbon monoxide in the synthesis gas is reacted with the dimethyl carbonate to from oxygenated compounds such as methyl acetate, dimethyl ether (DME), and formaldehyde. A product containing methyl acetate, dimethyl ether, formaldehyde, unreacted dimethyl carbonate, carbon dioxide, unreacted synthesis gas, a portion of the catalyst, and a portion of the inert oil, is withdrawn from reactor 124 through line 125, and is passed to flash tank 126. In general, flash tank 126 is operated at a temperature of from about 100° C. to about 230° C., and at a pressure of from about 14 psi to about 900 psi. Flash tank 126 thus is operated under conditions such that the methyl acetate, dimethyl ether, formaldehyde, unreacted dimethyl carbonate, carbon dioxide, and unreacted synthesis gas are separated from the portion of the catalyst and the portion of the inert oil. The portion of the inert oil and the portion of the catalyst are withdrawn from flash tank 126 through line 127 and are recycled to reactor 124.

The product containing methyl acetate, dimethyl ether, formaldehyde, unreacted dimethyl carbonate, carbon dioxide, and unreacted synthesis gas is withdrawn from flash tank 126 through line 128, and is passed to flash tank 129. In general, flash tank 129 is operated at a temperature of from about 5° C. to about 50° C., and at a pressure of from about 14 psi to about 900 psi. Thus, flash tank 129 is operated under conditions such that the methyl acetate, dimethyl ether, formaldehyde, carbon dioxide, and unreacted synthesis gas are separated from any residual oil and unreacted dimethyl carbonate. The residual oil and unreacted dimethyl carbonate are withdrawn from flash tank 129 through line 131, passed through heater 132, and line 118, and then passed to mixing chamber 116.

Methyl acetate, dimethyl ether, formaldehyde, carbon dioxide, and unreacted synthesis gas are withdrawn from flash tank 129 through line 130, and passed to distillation column 133. In distillation column 133, the methyl acetate is separated from the dimethyl ether, formaldehyde, carbon dioxide, and unreacted synthesis gas, and is recovered through line 135.

Dimethyl ether, formaldehyde, carbon dioxide, and unreacted synthesis gas are withdrawn from distillation column 133 through line 134, and passed to cooling tower 136. In cooling tower 136, dimethyl ether and formaldehyde are separated from the carbon dioxide and unreacted synthesis gas. The dimethyl ether and formaldehyde are recovered from line 138. Carbon dioxide and unreacted synthesis gas are withdrawn from cooling tower 136 through line 137. The unreacted synthesis gas may, if desired, be separated from the carbon dioxide and recycled to compressor 121.

The disclosures of all patents and publications, including published patent applications, are incorporated herein by reference to the same extent as if each patent and publication were incorporated individually by reference.

It is to be understood, however, hat the scope of the present invention is not to be limited to the specific embodiments described above. The invention may be practiced other than as particularly described and still be within the scope of the accompanying claims.

What is claimed is:

1. A method of producing at least one oxygenated compound selected from the group consisting of methyl acetate, dimethyl ether, formaldehyde, and mixtures thereof, comprising:
reacting dimethyl carbonate with carbon monoxide to convert at least a portion of said dimethyl carbonate and a portion of said carbon monoxide to said at least one oxygenated compound, wherein said dimethyl carbonate and said carbon monoxide are reacted in the presence of at least one solid catalyst suspended in an inert liquid.

2. The method of claim 1 wherein said at least one solid catalyst is at least one zeolite catalyst.

3. The method of claim 2 wherein said at least one zeolite catalyst s selected from the group consisting of faujasite zeolites, zeolite Beta, Linde Type L (LTL) zeolite, MCM-41, mordenite, zeolite X, zeolite Y, ZSM-5, and mixtures thereof.

4. The method of claim 3 wherein said at least one zeolite is a faujasite zeolite.

5. The method of claim 3 wherein said at least one zeolite is zeolite Beta.

6. The method of claim 3 wherein said at least one zeolite is LTL zeolite.

7. The method of claim 3 wherein said at least one zeolite is MCM-41.

8. The method of claim 1 wherein said at least one oxygenated compound is methyl acetate.

9. The method of claim 1 wherein said carbon monoxide is obtained from synthesis gas.

10. The method of claim 1 wherein said inert liquid is an inert oil.

11. The method of claim 10 wherein said inert oil is a white mineral oil.
